(19) United States
(12) Patent Application Publication
(10) Pub. No.: US 2009/0048547 A1
(43) Pub. Date: Feb. 19, 2009

(54) Hand massager

(76) Inventor: Samuel Chen, Hunghorn Kowloon (HK)

Correspondence Address: LAW OFFICES OF CLEMENT CHENG, 17220 NEWHOPE STREET #127, FOUNTAIN VALLEY, CA, 92708, US

(21) Appl. No.: 11/891,993

(22) Filed: Aug. 14, 2007

(51) Int. Cl. A61N 1/00; A61H 7/00

(52) U.S. Cl. 601/15; 601/46; 601/84

(57) ABSTRACT

A hand massager has a base comprising a plurality of movable massage knobs and a swivel connection, wherein the base is shaped to receive a user's hand. The hand massager also has a top connected to the swivel connection. The top is sized to fit over a user's hand to define a cavity between the top and the base. Electronic user controls controlling the motion of movable massage knobs. The top may further include a vibrating element for massaging the top of a user's hand. The top can also have a heat element. The base has a heat element. The massage knobs, vibrating elements and heat elements interact with each other to make a hand massager that is desktop friendly and can be used in a home environment.

### DISCUSSION OF RELATED ART

Hand massaging is well known in acupressure therapy. A number of hand massaging techniques such as from shiatsu have benefited a large number of people who have suffered from tendonitis and hand pain. Hand massage has become more popular with increased keyboard usage at the office. Hand massage can also be relaxing. Hand massage can invigorate tired fingers and sore tendons. Many people enjoy a hand massage after a long day of work. Unfortunately, massaging a hand is hard work itself and therefore, it is an object of the present invention to provide a mechanical desktop hand massager that is relaxing and can be used in the comfort of one's home. Hand massage requires a variety of movements including making small circular movements among the tendons, rubbing the palm and squeezing the web area between fingers. Thus, a good hand massage requires a variety of complex movements.

There are a variety of manual hand massaging techniques that alleviate overworked and overstrained hands. Pressing the back of the hand with fingers and massaging between the tendons of the fingers helps stretch tendons and muscles. Also, the soft area on the palm below the palm and below each knuckle can also be massaged. The wrist can also be massaged by placing the fingers on top of the wrist and pressing down. Sometimes oil is applied to alleviate uncomfortable friction. Sometimes hand massage is followed by stretching and interlocking fingers.

A variety of areas on the hand require many small motor hand movements and may be tired. For example the web area between the thumb and index finger may be a good place to massage. This area has been called the ‘hoku’ point in acupressure and shiatsu massage. Breathing deeply also improves relaxation while applying hand massaging techniques. Other movements may include pressing a first into the palm and applying firm pressure. Also, the thumb can be pressed into the palm of the hand.

In any case, using your own tired hands to massage your hands seems to be kind of counterproductive since hand massage is fairly hand labor intensive. Therefore it is a present object of the invention to have a mechanical hand massager so that people do not have to use their own tired hands to massage their hands.

### BRIEF DESCRIPTION OF THE DRAWINGS

FIG. 1 is a perspective view of the hand massager in closed position.

FIG. 2 is a side view of the hand massager in open position.

FIG. 3 is a plan view of the base of the hand massager.

FIG. 4 is a second embodiment of the hand massager.

FIG. 5 is a perspective view of the second embodiment of the hand massager which lacks the swivel connection.

FIG. 6 is a side view of the second embodiment of the hand massager showing the airbags.

FIG. 7 is a top view of the second embodiment.

### DETAILED DESCRIPTION OF THE PREFERRED EMBODIMENT

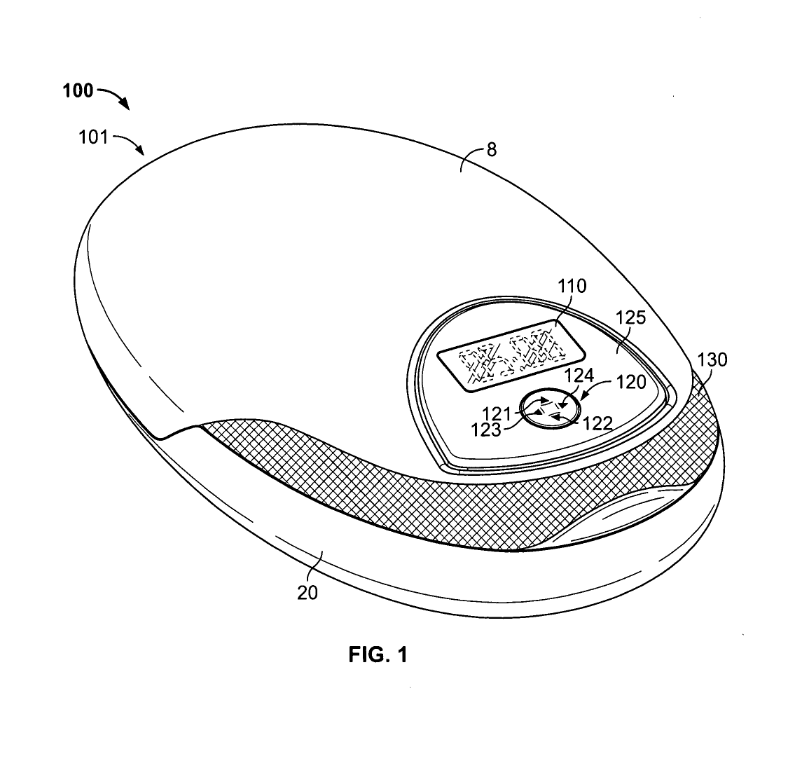

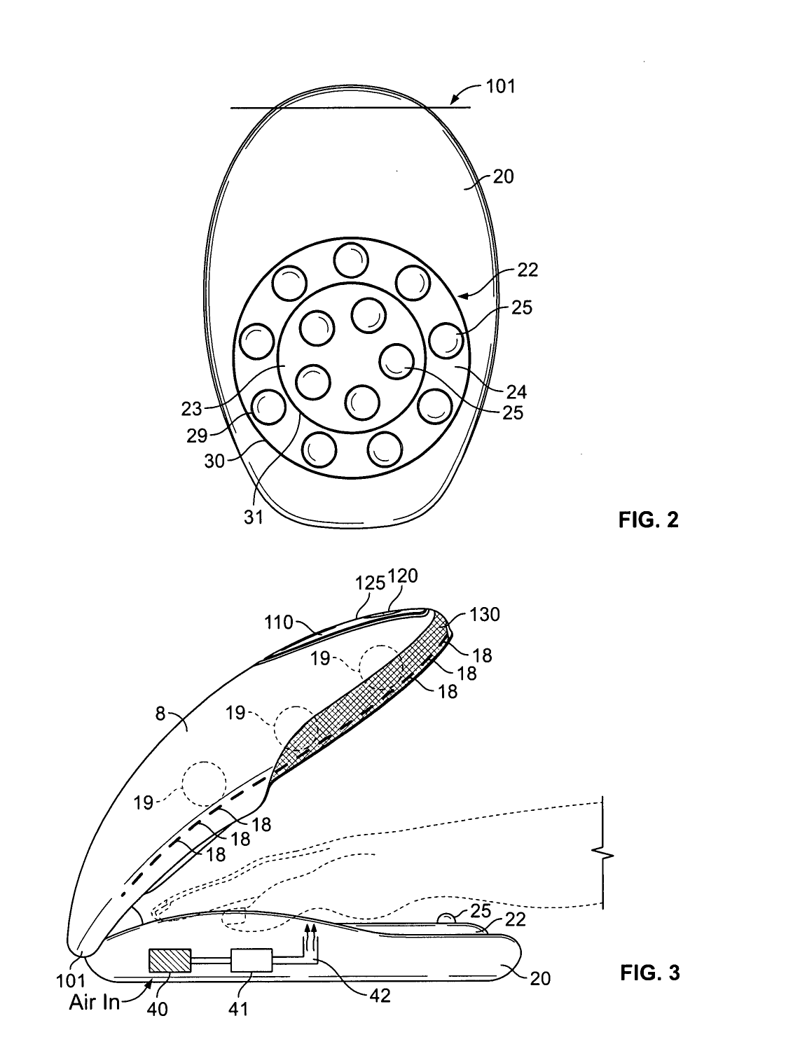

FIG. 1, a hand massager device 100 has a base portion 20 and a top portion 8. The top preferably swivels with the base so that it opens in a clamshell fashion allowing a user to place a hand within a hand massaging area formed between the top and the base. The swivel mechanism 101 is preferably a pivot such as a pin joint. The rear portion of the top and the rear portion of the base can be connected by a pivot joint. The hand massager therefore has a closed position FIG. 1 and an open position FIG. 2. The total size of the hand massager should be small enough to allow desktop use. The hand massager is electrically powered and can be plugged into household electric current. The electric power operates the heating elements and the vibrating elements such as knobs 19.

The hand massager has an opening for the wrist. The opening for the wrist allows the top to close against the bottom forming a hand massaging cavity between. The area around the wrist opening preferably has soft padding for maximum wrist comfort.

The top further comprises a heat blanket for heating the top of the hand. A heat blanket can also be placed in the base for heating the palm of the hand. The heat blanket is preferably resistance heated with thermostat control. Preferably, the user can use the controls 120 and set the temperature of the heat element for a specific temperature, or a relative one such as hot, warm or the like. The controls can be formed as a set of buttons mounted on the top 8 of the device 100. The buttons can include a set of time setting buttons, and temperature setting buttons. The buttons can be configured as rockers with a pair of positive and negative matching buttons in opposing connection. The up button 121 and down button 122 can control either the time or temperature and the left button 123 with the right button 124 can also control either the time or temperature.

Optionally, the display 110 can show the set temperature such as 85° Fahrenheit or warm, or a number such as five on the scale of one to five. The display 110 can also show a time for which the temperature is set for. The heat blanket setting can expire after a certain time and the countdown of the time setting can also be shown on the display 110. Preferably, the display 110 is a liquid crystal flat panel display. It is optionally backlit for nighttime use. The display is preferably located on the operations panel 125.

The heat blanket 18 can be filled with thermally retentive beads, or some other type of thermally retentive filler. Also, the heat blanket is preferably a soft fabric that may have a gel or soft padding for maximum hand comfort. With the heat blanket formed as a variety of separate elements that individually get hot, the heat blanket may have a warmer front portion facing the user and a cooler rear portion away from the user toward the user fingertips. With separate elements, the user fingers and palm can receive varying heat blanket and heat treatment.

The top further comprises vibration elements. Vibration elements such as knobs 19 can also mount in the base next to the base heat element. The vibration elements can be formed as round vibration knobs that have a motor mechanically attached to the knobs. The round vibration knobs can be formed as spheres as shown in FIG. 2 of the preferred embodiment.

The knobs 19 vibrate upon activation. Vibration frequency and amplitude can be controlled using user controls. The knobs are the vibration elements 19. The vibration elements 19 can be driven by electric motor spinning an eccentric mass creating vibration. Optionally, the motor and eccentric mass can be tuned for harmonic vibration. The knobs may be formed as hollow plastic shells having a round surface facing a user hand. The motor and eccentric mass may be placed within the shell, or otherwise rigidly mounted to the hollow plastic shell. The entire unit of the shell, motor and eccentric mass is the knob which is a type of vibration element.

The bottom portion which is the base 20 can also have massaging knobs 25. FIG. 3 of the preferred embodiment shows a number of massaging knobs mounted on a circular platform 22 in the hand massaging cavity. The circular platform optionally embodied as a round rotor 22 rotates in various directions to massage the palm of the user. The circular platform can be formed as a single sheet of plastic having round protrusions. The circular platform can also be formed independently of the massaging knobs 25. In either case, the massaging knobs optionally vibrate to provide massage to a user palm. A number of massage knobs are mounted in the top and base of the hand massager. The massage knobs have motors mechanically attached, or located within allowing the massage knobs to vibrate against a hand to provide a vibration massage. The circular platform 22 is placed and sized so that it massages the palm of a user.

The circular platform 22 may have an outside section 24 formed as a ring holding the massaging knob 25 and an inside section 23 formed as a circle. The inside section 23 and outside section 24 can rotate in a variety of directions, or randomly to provide a variety of massaging sensations on the palm of the user. An inside gap 31 between the inside section 23 and the outside section 24 can provide air flow outlet to the user palm. Similarly, an outside gap 30 formed between the outside section 24 and the lower shell of the base 20 can also provide airflow outlet to the user palm.

Air can be pumped through an air inlet 40 by an air pump 41 which has an air conduit 42 that sends air to the palm of a user such as by air outlet outside gap 30 or inside gap 31. Alternatively, instead of the air outside gap 30 or inside gap 31 providing airflow to the user palm, airflow can be provided by openings in the circular platform 22 such as on the inside section 23 or the outside section 24. The air then leaves the device by the gap formed between the upper portion 8 and the base 20.

A variety of displays 110 show time and settings. Controls 120 on the housing allow a user to control the heat and vibration modes for the hand massager. Preferably, the displays and controls are located on the upper surface of the top. The controls can activate the massage for a set time, activate the massage intermittently, and also control temperature.

A soft padded region 130 preferably covers the surfaces touched by a user. The top can close over the base, optionally locking the hand and pressing the hand toward the massage knobs. The swivel connection can also have a spring such as a helical spring that biases the top against the base. The strength of the helical spring can be adjusted to allow for a range of press force. The bottom surface of the top is preferably spring biased so that when the top is closed, the spring bias pushes the heat blanket and vibration elements against the top of the hand. The vibration elements are preferably spring biased away from the top.

The bottom of the device 100 preferably has a base 20 grippy bottom surface such as individual feet formed as posts for a sturdy surface connection with a table. The torque generated by the round rotor 22 should be balanced by a base 20 grippy bottom surface.

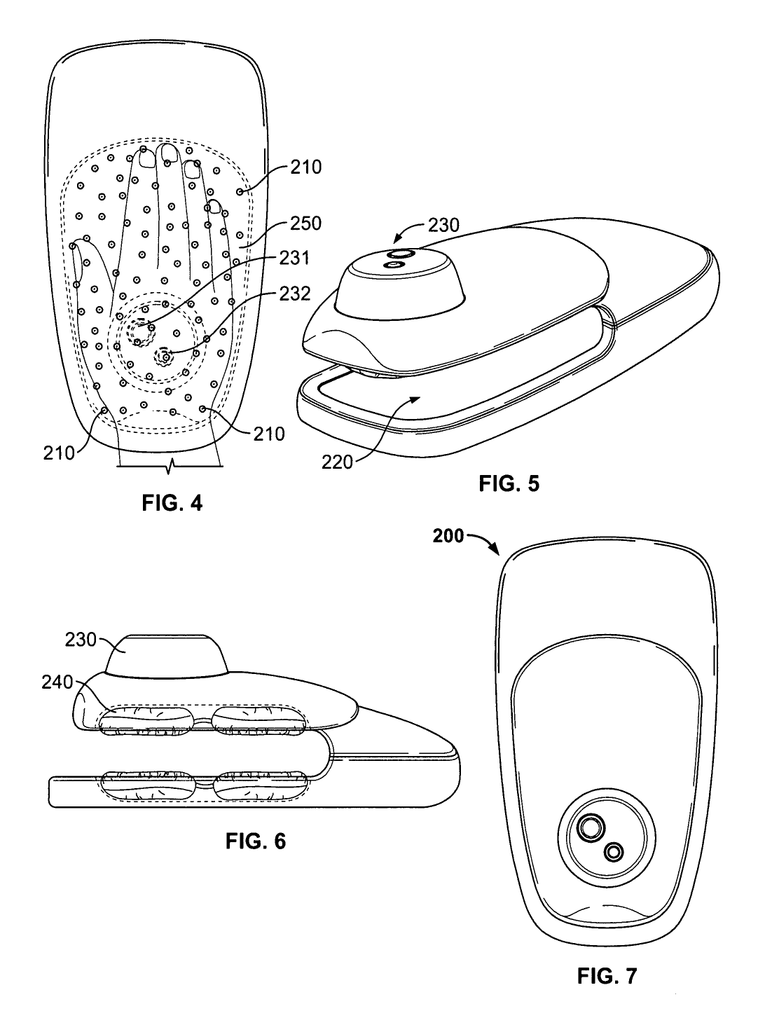

In a second embodiment of the device shown in FIG. 4, the device does not have the swivel connection but simply has an upper portion opposing a lower portion. A plurality of massaging knobs massage the top of a hand in the second embodiment. An airbag adds pressure to the top and bottom portions so that the user's hand receives an airbag massage.

A number of upper small massaging knobs 210 are evenly spaced and press against the tissues on the hand. The hand is placed inside a hand massaging area forming a shallow depression 250. The upper small massaging knobs 210 are preferably placed so that they substantially cover the area of a persons hand. The upper small massaging knobs 210 can be mounted on the airbags 240. A vibration mechanism optionally agitates the airbags and gives greater motion to the upper small massaging knobs 210. Otherwise, the upper small massaging knobs 210 cushion and press against the hand as the airbags 240 expand.

The cycle of airbag inflation and deflation provides circulatory benefit for a user hand. As the airbags 240 expand, with the upper small massaging knobs 210 press on tendons and muscles. As the airbags 240 contract, the small massaging knobs 210 move away from the user's hand. As seen in FIG. 5, a top rotating knob 230 causes a motor housing protrusion seen on the top of the device. The top rotating knob 230 gives the user a rotating massage from a pair of rollers 231, 232 mounted on the top rotating knob 230. As the top rotating knob 230 rotates, the user receives a hand massage on the back of the hand by rollers 231 and 232. The top rotating knob 230 can also be in sliding configuration with the device housing so that a user may place the other hand not being massaged on top of the top rotating knob 230 so that the user can increase the pressure on the massaged hand. The top rotating knob 230 also shows a circular rotating platform as seen in FIGS. 4, 5 and 7.

A cross-section of the device as shown in FIG. 6 shows the location of the airbags. The preferred method is to have a pair of lower airbags in opposition to a pair of upper airbags. The gap 220 provides an opening for a user hand. The user hand fits comfortably within the gap 220. When the airbags 240 inflate, the gap 220 shrinks accordingly. By having a pair of forward airbags and a pair of rear airbags, the pressure of the front airbags can be varied so that they are greater or less than the pressure of the rear airbags. The front airbags correspond to pressure on the fingers and the rear airbags correspond to pressure on the palm and optionally wrists. By having a pair of upper airbags and a pair of lower airbags, the upper airbags can be inflated so that they are greater or less than the pressure of the lower airbags. Optionally, the four airbags are joined so that they are in fluid communication with each other. The airbags can be filled with water, air or a mixed fluid.

Also, the elements of the second embodiment can be combined with the elements of the first embodiment. For example, the massaging plate having the inside and outside rotating knobs 25 of the first embodiment, can be mounted in the second embodiment housing such as in the bottom part of the gap 220 as a substitute for the lower airbags. Also, the heated blanket element 18 of the first embodiment can be incorporated into the acupressure knob 210 element of the second embodiment to have warmed acupressure knobs 210. Yet another example is providing the air blower 40 and the air conduit 42 in the lower portion of the second embodiment so that the lower pair of airbags have spaces between them where air blows out to the user palm.

The following call out list of elements is for assistance in matching the call out numbers on the drawings with the specification.

| | |
|---|---|
| 100 | Device |
| 101 | Swivel Connection |
| 110 | Time And Setting Display |
| 120 | Control |
| 130 | Soft Padding |
| 8 | Top |
| 18 | Heat Blanket |
| 19 | Vibration Elements |
| 20 | Base |
| 22 | Round Rotor |
| 25 | Massage Knob |

The foregoing describes the preferred embodiments of the invention. Modifications may be made without departing from the spirit and scope of the invention as set forth in the following claims. The present invention is not limited to the embodiments described above, but encompasses any and all embodiments within the scope of the following claims. For example, the housing is shown as a soft curvature rounded style housing, but can be replaced with an angular square or rectangular style housing. Also, the digital LCD readout 10 could be replaced by a suitable OLED, LED or other type of display panel that can display characters.

1. A hand massager comprising:
a. a base comprising:
i. a plurality of movable massage knobs;
ii. a swivel connection, wherein the base is shaped to receive a user's hand;
b. at least one upper airbag mounted above the cavity and one lower airbag mounted below the cavity;
c. a top connected to the swivel connection, the top sized to fit over a user's hand to define a cavity between the top and the base, wherein the cavity is a hand cavity; and
d. electronic user controls controlling the motion of movable massage knobs.

2. The hand massager of claim 1, wherein the top further comprises at least one vibrating element for massaging the top of a user's hand.

3. The hand massager of claim 2, wherein the top further comprises a heat element.

4. The hand massager of claim 3, wherein the base has a heat element.

5. The hand massager of claim 1, wherein the top further comprises a heat element.

6. The hand massager of claim 5, wherein the base has a heat element.

7. The hand massager of claim 1, wherein the base has a heat element.

8. A hand massager comprising:
a. a base comprising: a plurality of movable massage knobs mounted on a circular platform;
b. a top connected to the swivel connection, the top sized to fit over a user's hand to define a cavity between the top and the base;
c. an upper airbag mounted above the cavity and a lower airbag mounted below the cavity, wherein the cavity is a hand cavity;
d. electronic user controls controlling the motion of movable massage knobs and the upper airbag and the lower airbag.

9. The hand massager of claim 8, wherein the top further comprises at least one vibrating element for massaging the top of a user's hand.

10. The hand massager of claim 9, wherein the top further comprises a heat element.

11. The hand massager of claim 10, wherein the base has a heat element.

12. The hand massager of claim 11, wherein the top further comprises a heat element.

13. The hand massager of claim 12, wherein the base has a heat element.

14. The hand massager of claim 8, wherein the base has a heat element.

15. A hand massager comprising:
a. a base comprising: a plurality of movable massage knobs mounted on a circular platform;
b. a top connected to the swivel connection, the top sized to fit over a user's hand to define a cavity between the top and the base;
c. a pair of upper airbags mounted above the cavity and a pair of lower airbags mounted below the cavity, wherein the cavity is a hand cavity;
d. electronic user controls controlling the motion of movable massage knobs and the pair of upper airbags and the pair of lower airbags; and
e. an air pump for pumping air on the palm.

16. The hand massager of claim 15, wherein the top further comprises at least one vibrating element for massaging the top of a user's hand.

17. The hand massager of claim 16, wherein the top further comprises a heat element.

18. The hand massager of claim 15, wherein the base has a heat element.

19. The hand massager of claim 15, wherein the top further comprises a heat element.

20. The hand massager of claim 19, wherein the base has a heat element.
